(19) United States
(12) Patent Application Publication
(10) Pub. No.: US 2012/0141589 A1
(43) Pub. Date: Jun. 7, 2012

(54) PARTICLES FOR DRUG DELIVERY AND OTHER APPLICATIONS

(75) Inventors: Ben Fan, Somerville, MA (US); Roland S. Koltzenburg, Dannstadt (DE); Jens B. Rieger, Ludwigshafen (DE); David A. Weitz, Bolton, MA (US)

(73) Assignees: BASF SE, Ludwigshafen (DE); President and Fellows of Harvard College, Cambridge, MA (US)

(21) Appl. No.: 13/215,819

(22) Filed: Aug. 23, 2011

Related U.S. Application Data
(60) Provisional application No. 61/376,149, filed on Aug. 23, 2010.

(51) Int. Cl. A61K 31/565; A61K 9/14; A61K 31/58

(52) U.S. Cl. 424/489; 514/182; 514/176

(57) ABSTRACT

The present invention generally relates to particles for drug delivery and other applications. In one aspect, the present invention relates to a technique for reacting precursor compounds in the presence of a pharmaceutically-active agent to form product (e.g., in the form of particles) in which the agent is substantially contained within the product, and the product is soluble within typical gastric fluid of a mammal. In another aspect, the present invention is generally directed to particles comprising an inorganic pharmaceutically acceptable carrier, such as CaCO₃, and an agent. In some cases, at least some of the agent contained within the particles is fluidically inaccessible from externally of the particle. For instance, the agent may be present in isolated domains within the particle. In another aspect, the present invention is generally directed to methods of creating particles. For instance, according to one set of embodiments, two fluids containing reactants are mixed where, upon reaction of the reactants, an insoluble product is formed, which precipitates to form particles. In one example, a first fluid containing dissolved carbonate ions and a second fluid containing dissolved calcium ions and a pharmaceutically-active agent are mixed together; upon mixing of the first and second fluids, the calcium ions and the carbonate ions form calcium carbonate, which precipitates to form a co-precipitate with the pharmaceutically-active agent. Yet other aspects of the present invention are directed to particles formed from such reactions, methods of using such reactions, methods of promoting such reactions, kits involving particles, or the like.

### RELATED APPLICATIONS

This application claims the benefit of U.S. Provisional Patent Application Ser. No. 61/376,149, filed Aug. 23, 2010, entitled “Particles for Drug Delivery and Other Applications,” by Fan, et al., incorporated herein by reference.

### FIELD OF INVENTION

The present invention generally relates to particles, including particles for drug delivery and other applications.

### BACKGROUND

CaCO₃ is often used in oral drug delivery formulations because of its solubility in acid. For instance, a formulation containing CaCO₃ will readily dissolve upon exposure to the acid of the stomach (typically at pHs of 2 or less). Accordingly, active agents contained within CaCO₃ formulations will not be released after ingestion until exposed to stomach acid. However, due to the insolubility of CaCO₃ in water at neutral pH, it can be difficult to introduce certain types of agents in CaCO₃ formulations. Thus, new techniques for improving the loading of formulations comprising CaCO₃ and similar compounds are still needed.

### SUMMARY OF THE INVENTION

The present invention generally relates to particles for drug delivery and other applications. The subject matter of the present invention involves, in some cases, interrelated products, alternative solutions to a particular problem, and/or a plurality of different uses of one or more systems and/or articles.

In one aspect, the present invention is directed to a method. According to a first set of embodiments, the method includes acts of providing a first fluid comprising a first solvent and a first reactant; providing a second fluid comprising a second solvent, a second reactant, and a pharmaceutically-active agent; and mixing the first fluid and the second fluid to form a mixed fluid. In some cases, the second fluid and the first fluid are substantially miscible. In certain embodiments, the first reactant interacts with the second reactant to form an inorganic product that is insoluble in the mixed fluid but is soluble in aqueous-based solution at pH of less than 4. The pharmaceutically-active agent may be substantially insoluble in the mixed fluid in some embodiments.

In another set of embodiments, the method includes acts of providing a first fluid comprising a first solvent and a first reactant; providing a second fluid comprising a second solvent, a second reactant, and an agent contained in the second fluid; and mixing the first fluid and the second fluid to form a mixed fluid. In some cases, the second fluid and the first fluid are substantially miscible. The first reactant, in some embodiments, may interact with the second reactant to form a non-polymerized product that is insoluble in the mixed fluid. According to some embodiments, the agent may be insoluble in the mixed fluid.

Another aspect of the invention is generally directed to a composition. In one set of embodiments, the composition includes a comprising or consisting essentially of one or more carbonates and a hydrophobic pharmaceutically active agent. In some embodiments, at least some of the agent is fluidically inaccessible from externally of the particle.

In another set of embodiments, the composition includes a particle comprising or consisting essentially of one or more carbonates and an agent. In some cases, the agent is present in isolated domains within the particle. The isolated domains may have, in some embodiments, a largest dimension of no more than about 1 micrometer.

The composition, according to yet another set of embodiments, includes particles substantially each comprising or consisting essentially of one or more carbonates and an agent. The particles, in some cases, exhibit an apparent release rate of the agent that is at least about 50% greater than an apparent release rate of the agent from a control material having the same overall composition and average diameter as the particles. The control material may consist essentially of homogeneous particles, at least in some embodiments.

In another aspect, the present invention is directed to a method of making one or more of the embodiments described herein, for example, a particle comprising CaCO₃. In another aspect, the present invention is directed to a method of using one or more of the embodiments described herein, for example, a particle comprising CaCO₃.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B are photomicrographs of various particles in accordance with one embodiment of the invention;

FIGS. 2A-2B illustrate EDAX analysis of particles according to another embodiment of the invention;

FIG. 3 illustrates UV absorption of dissolved particles, in accordance with still another embodiment of the invention;

FIG. 4 is a schematic diagram showing separate domains within a particle, in yet another embodiment of the invention;

FIGS. 5A and 5B illustrate SEM images of particles produced in accordance with one embodiment of the invention;

FIG. 6 illustrates XRD data of particles in an embodiment of the invention;

FIG. 7 illustrates a Raman spectrum of particles in another embodiment of the invention;

FIGS. 8A-8B illustrate Raman confocal micrographs of particles in an embodiment of the invention; and

FIGS. 9A-9C illustrate dissolution rates for certain particles produced in accordance with various embodiments of the invention.

### DETAILED DESCRIPTION

The present invention generally relates to particles for drug delivery and other applications. In one aspect, the present invention relates to a technique for reacting precursor compounds in the presence of a pharmaceutically-active agent to form product (e.g., in the form of particles) in which the agent is substantially contained within the product, and the product is soluble within typical gastric fluid of a mammal. In another aspect, the present invention is generally directed to particles comprising an inorganic pharmaceutically acceptable carrier, such as CaCO₃, and an agent. In some cases, at least some of the agent contained within the particles is fluidically inaccessible from externally of the particle. For instance, the agent may be present in isolated domains within the particle. In another aspect, the present invention is generally directed to methods of creating particles. For instance, according to one set of embodiments, two fluids containing reactants are mixed where, upon reaction of the reactants, an insoluble product is formed, which precipitates to form particles. In one example, a first fluid containing dissolved carbonate ions and a second fluid containing dissolved calcium ions and a pharmaceutically-active agent are mixed together; upon mixing of the first and second fluids, the calcium ions and the carbonate ions form calcium carbonate, which precipitates. In some cases, the precipitant may also contain an agent, such as a pharmaceutically-active agent. The agent may be present in the second fluid in a dissolved state, but upon mixing of the first fluid and the second fluid, the agent cannot stay in a dissolved state, and thus precipitates. Yet other aspects of the present invention are directed to particles formed from such reactions, methods of using such reactions, methods of promoting such reactions, kits involving particles, or the like.

A first aspect of the present invention is generally directed to two or more fluids that, when mixed, result in the formation of one or more precipitants. In certain embodiments, more than one precipitant is formed, and the various precipitants may “co-precipitate” together (i.e., precipitating at the same time and/or due to the same fluidic conditions), for example, resulting in solid particles formed from some or all of the precipitants. As discussed in detail herein, such fluids and systems can be advantageously used in the preparation of certain formulations for drug delivery and other applications. In one set of embodiments, for instance, a first fluid comprising a first solvent and a first reactant is mixed with a second fluid comprising a second solvent and a second reactant. The solvent is able to contain the reactant, e.g., by dissolving the reactant, or carrying the reactant as a dispersion or a suspension, etc. The first solvent and the second solvent may be substantially miscible, such that mixing the first solvent and the second solvent results in a non-phase separated (or “homogeneous”) mixture of the first solvent and the second solvent, rather than a phase-separated system (e.g., as in an emulsion or a partitioned liquid-liquid system). The first solvent and the second solvent may each be, for example, both hydrophilic. Non-limiting examples of hydrophilic solvents include alcohols, such as ethanol, methanol, 1-propanol, 2-propanol, or the like. Other examples of hydrophilic solvents include, but are not limited to, 1,2-butanediol, ethylene glycol, propylene glycol, glycerol, and/or water (i.e., producing an aqueous solution when water is used as a solvent). Still other examples include polar aprotic solvents such as tetrahydrofurane, acetone, dimethyl sulfoxide, N,N-dimethylformaide, or the like; acidic compounds such as formic acid or acetic acid, etc.; ethers such as glycol dimethyl ether, diglycol dimethyl ether, glycol methyl ether, diglycol methyl ether, 1-methoxy-2-butanol, etc. It should be understood that, as used herein, a “fluid” is intended to include not only a pure species, but also mixtures of two or more species, each of which may be present in any form and in any concentration. For example, a fluid may consist essentially of water, water containing dissolved or suspended salts or other compounds, a mixture of water and ethanol, a mixture of water and ethanol containing dissolved or suspended salts or other compounds, etc. Thus, the first fluid and the second fluid may each independently be a solution (e.g., an aqueous solution), a suspension (e.g., a colloidal suspension), a dispersion, or the like. The first fluid and the second fluid may have the same or different compositions. For example, a first fluid may consist essentially of water while a second fluid comprises a mixture of water and ethanol.

In one set of embodiments, one or both of the fluids is hydrophilic. As used herein, a “hydrophilic” fluid is a fluid that is substantially miscible in water, at least at ambient temperature (25° C.) and pressure (1 atm), such that upon mixing of the hydrophilic fluid and water, no substantial phase separation is observed over a time of at least a day. (It should be noted, of course, that water is completely miscible in itself, thus water is a hydrophilic fluid) In some embodiments, the hydrophilic fluid may be substantially miscible in water at elevated temperatures and/or pressures. For example, the hydrophilic fluid may be substantially miscible in water at temperatures of at least about 50° C., at least about 75° C., at least about 100° C., at least about 125° C., at least about 150° C., at least about 175° C., or at least about 200° C. Relatively higher temperatures (e.g., at least about 100° C.) may be achieved, for example, at elevated pressures, e.g., pressures of at least about 2 atm, at least about 3 atm, at least about 4 atm, at least about 5 atm, at least about 6 atm, at least about 8 atm, at least about 10 atm, at least about 12 atm, at least about 14 atm, at least about 16 atm, etc.

The first reactant and the second reactant in the respective first and second fluids may react upon mixing of the fluids to form a product that is substantially insoluble in the mixture of the first fluid and the second fluid, i.e., the product forms a separate phase that can precipitate or otherwise separate from the mixture of the first fluid and the second fluid. The product may be a solid in some cases, and in certain embodiments, the product precipitates from the mixture as solid particles. The reaction may be any suitable chemical reaction including, for example, an ion exchange reaction.

In one set of embodiments, the reaction may be a single displacement reaction (e.g., where A+BX→AX+B, each letter representing an ion) or a double displacement reaction (e.g., where AX+BY→AY+BX); one of these products may be substantially insoluble in the mixture of the first fluid and the second fluid, and can be recovered as a separated phase or precipitant. In some cases, the reactions may be ionic reactions, where the first reactant (i.e., A or AX, respectively) is present in a dissolved state in a first fluid and the second reactant (i.e., BY or BX, respectively) is present in a dissolved state in a second fluid.

The product formed from the mixture of the first fluid and the second fluid may be a polymer, an inorganic compound such as an inorganic salt, or the like. In one set of embodiments, however, the product is not a polymer. In some cases, the product may be one with a relatively low molar mass (i.e., molecular weight), e.g., of less than about 1000 Da (g/mol), less than about 500 Da, less than about 300 Da, less than about 200 Da, less than about 150 Da, or less than about 100 Da. An inorganic compound is one that does not contain any C—H covalent bonds, although in some cases, the inorganic compound may contain carbon atoms, such as CaCO₃, and/or hydrogen atoms, such as HCl, Ca(HCO₃)₂, or H₂CO₃.

As a specific non-limiting example, a first fluid containing carbonate ions and a second fluid containing calcium ions may be mixed together, where the carbonate ions and the calcium ions combine to form CaCO₃, which under some conditions will precipitate. Other ions may be used instead of or in addition to calcium ions, for example, magnesium ions, sodium ions, potassium ions, silicon ions, or the like. Carbonate and/or other ions may be introduced into the first fluid using any suitable technique. For instance, carbonate salts such as Na₂CO₃, K₂CO₃, or (NH₄)₂CO₃, NaHCO₃, KHCO₃, (NH₄)HCO₃, etc. may be dissolved in the first fluid, or salts such as CaCl₂ (optionally in the form of a hydrate such as CaCl₂.2H₂O), Ca(NO₃)₂, calcium acetate, MgCl₂, Mg(NO₃)₂, magnesium acetate, NaCl, Na₂CO₃, NaNO₃, sodium acetate, KCl, K₂CO₃, KNO₃, potassium acetate, etc. may be dissolved in the second fluid. In one set of embodiments, the precipitate will consist essentially of calcium carbonate and an agent. In another set of embodiments, the precipitate will comprise more than one carbonate (for example, one or more of calcium carbonate, magnesium carbonate, sodium carbonate, potassium carbonate, etc.) and the agent.

Combinations of more than one of these salts and/or other salts may independently be present in each respective fluid in some cases. As examples, the first fluid may be water, ethanol, or another hydrophilic fluid, and the second fluid may independently be water, ethanol, or another hydrophilic fluid.

The separated phase or precipitant may include an agent that is present in one (or both) of the first fluid and the second fluid. For instance, in some cases, an agent may be dissolved or suspended within the second fluid, but the agent may be relatively insoluble in the mixture of the first fluid and the second fluid, and the agent can precipitate or otherwise form a separate phase from the mixture of the two fluids. Thus, upon mixing of the first fluid and the second fluid, the agent is no longer able to stay dissolved or suspended, and instead precipitates or forms a separate phase from the mixture of the two fluids, e.g., a solid phase. Such behavior can unexpectedly be used to cause the formation of a co-precipitate with a product formed from the first and second reactants, as described above, e.g., in particles. Accordingly, care must be used in selecting suitable first and second fluids, where the agent is soluble in the second fluid, but not in a mixture of the first and second fluids.

As a specific non-limiting example, in one set of embodiments, the agent may be one that is relatively insoluble in water, e.g., having a solubility in water at 20° C. of less than about 10 g/l. In some cases, however, the agent may be more soluble in other solvents, such as ethanol or methanol, where these solvents are hydrophilic and substantially miscible with water. For instance, the agent may be dissolved or suspended in ethanol, and when ethanol is mixed with water, the agent cannot stay in a dissolved or suspended state in the mixture of ethanol and water, and thus may precipitate or otherwise form a separate phase, e.g., a solid phase. Non-limiting examples of such agents include estradiol, danazol, or fenofibrate. In one set of embodiments, the agent is hydrophobic, i.e., at 20° C. and 1 bar, the agent has a solubility in pure water of less than about 10 g/l, less than about 5 g/l, less than about 3 g/l, less than about 1 g/l, less than about 500 mg/l, less than about 300 mg/l, less than about 100 mg/l, less than about 50 mg/l, less than about 30 mg/l, less than about 10 mg/l, etc. The agent may be used in applications such as pharmaceutical applications, nutritional applications, cosmetic applications, crop protection formulations, or the like.

During mixing, in some cases, the concentrations of the first and second reactants may be selected to avoid excess dilution or slow reaction. For example, the concentration of the first reactant in the first fluid may be at least about 5 wt %, at least about 10 wt %, at least about 15 wt %, at least about 20 wt %, at least about 25 wt %, at least about 30 wt %, at least about 35 wt %, at least about 40 wt %, at least about 45 wt %, or at least about 50 wt %, etc. Similarly, the concentration of the second reactant in the second fluid may be at least about 5 wt %, at least about 10 wt %, at least about 15 wt %, at least about 20 wt %, at least about 25 wt %, at least about 30 wt %, at least about 35 wt %, at least about 40 wt %, at least about 45 wt %, or at least about 50 wt %, etc. The concentrations may be selected independently of each other. In some cases, the concentrations are selected on a substantially stoichiometric basis. For instance, in some embodiments, the concentrations may be selected such that, upon mixing of the first fluid and the second fluid, at least 70 wt %, at least about 80 wt %, at least about 90 wt %, or at least about 95% of the reactants are able to react and/or precipitate, and in some cases under relatively short mixing times as is discussed below.

The precipitant formed by such reactions may precipitate in the form of particles. If an agent is present, in some cases, the agent may co-precipitate with the product formed from the first and second reactants, thereby forming particles comprising the agent and the insoluble product. The resultant particles are considered, in certain embodiments, to be a “solid dispersion” of the agent in the product formed from the first and second reactants. For example, the agent may be dispersed within the resulting product. In some cases, the agent and the product may be homogeneously mixed upon precipitation, e.g., at a molecular level; in other cases, however, the agent and product may form separate domains.

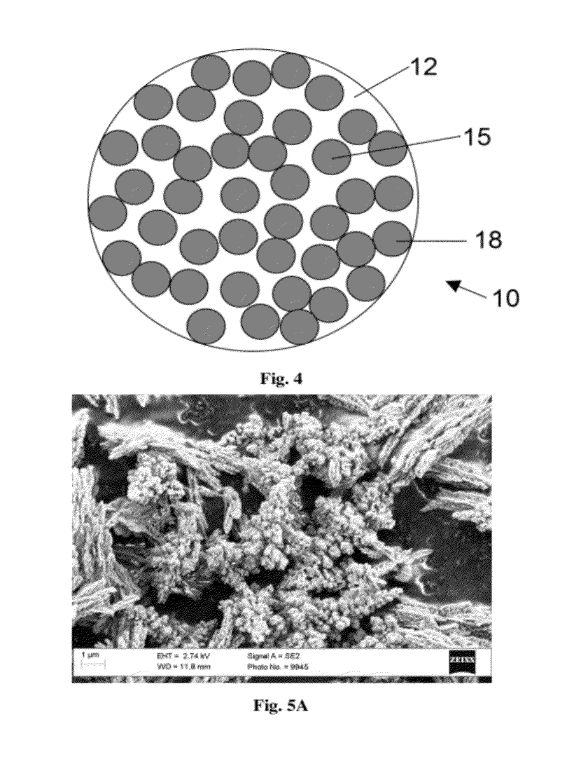

In some cases, the product and the agent co-precipitate to form particles where at least some of the agent is fluidically inaccessible from outside of the particles. Such arrangements can be achieved, for instance, due to the homogeneous nature of the co-precipitation of the product and the agent, where both are relatively well-mixed. In some cases, the agent and the product may form separate domains within the particle, but the separate domains are generally uniformly randomly distributed within the particle, thereby forming isolated domains in some cases, where at least some of the isolated domains of agent do not contact the outer surface of the particle. This is schematically illustrated in FIG. 4, which shows a cross section of a particle 10 formed from product 12 and an agent, which forms separate domains within the product in the particle, identified by 15 and 18. At least some of the domains in particle 10 are isolated from the outer surface of the particle, such as domain 15; however, there may be some domains that do contact the outer surface, such as domain 18.

Accordingly, in some embodiments, at least some of the agent may be present in isolated domains within the particle, where the isolated domains have a largest dimension of no more than about 10 micrometers, no more than about 5 micrometers, no more than about 3 micrometers, no more than about 1 micrometer, no more than about 500 nm, no more than about 300 nm, no more than about 200 nm, no more than about 100 nm, no more than about 50 nm, no more than about 30 nm, no more than about 20 nm, or no more than about 10 nm. In some cases, the size of the isolated domains within the particle may be such that they are identifiable using appropriate analytical techniques within the particles as isolated domains. It should be noted that the size of the isolated domains within the particle is independent of the size of the particle itself; particle sizes are discussed in more detail below. Relatively smaller isolated domains may, in some cases, be useful for decreasing dissolution rates of the domains, for example, to control the rate of release of the agent from the particle when the particle is dissolved. As a specific non-limiting example, a calcium carbonate particle containing a pharmaceutically acceptable agent present in relatively smaller isolated domains may be able to release the agent in a controlled manner once the particle has been ingested by a subject and is exposed to acidic gastric fluid able to dissolve calcium carbonate.

In some cases, the presence of isolated domains within a particle can be determined using fluid access tests. For example, a particle containing agent and product may be exposed to a test solvent able to dissolve the agent but unable to substantially dissolve the product; after sufficient exposure for the agent to be dissolved in the test solvent, the particle is removed from the solvent and analyzed in some fashion (e.g., destructively, by administration to a test subject if the agent has biological effects, etc.) to determine whether the particles still contains the agent or not, as agent present in isolated domains within the particles will not be exposed to the solvent, and cannot be removed from the particles under such conditions. In another set of embodiments, the presence of isolated domains within the particle may be determined by analytical techniques such as scanning electron microscopy (SEM), transmission electron microscopy (TEM), atomic force microscopy (AFM), or the like. For example, the particles may be embedded in a matrix and then microtomed into slices which are then analyzed using such techniques to determine the presence of isolated domains within the particles.

The agent may be present in any concentration within the particle. For example, at least about 5%, at least about 10%, at least about 15%, at least about 20%, at least about 25%, at least about 30%, at least about 35%, at least about 40%, at least about 45%, or at least about 50% of the mass of the particle may be formed by the agent. In some cases, the agent is present in an amount no more than can be carried by the second fluid used to form the particle. In certain embodiments, the agent may be present at a concentration of no more than about 60%, no more than about 50%, no more than about 40%, no more than about 30%, or no more than about 20% by mass within the particle.

One set of embodiments of the present invention is directed to particles formed from an inorganic compound, such as CaCO₃, and an agent such as described herein. Such particles may be used for applications such as drug delivery, e.g., oral drug delivery, or in other applications as discussed herein. For example, particles containing CaCO₃ may be dissolvable in acid environments or at low pHs, such as is found in the stomach (i.e., gastric acid), and as the particles dissolve, the agent may be released into the stomach. Thus, one embodiment of the invention is directed to particles comprising or consisting essentially of CaCO₃ and an agent. In some cases, such particles exhibit an apparent release rate of an agent that is at least about 50% greater than an apparent release rate of an agent from a physical mixture of separate homogenous particles having the same composition and the same size or size distribution. In some embodiments, the apparent release rate may be at least about 60% greater, at least about 80% greater, at least about 100% greater, at least about 150% greater, at least about 200% greater, at least about 300% greater, at least about 500% greater, etc., than an apparent release rate of an agent from a physical mixture of separate homogenous particles (e.g., not containing internal domains) having the same composition and the same size and/or size distribution. The release rate may be determined as the time it takes to release about 60%, about 70%, or about 80% of the agent from the particle.

Without wishing to be bound by any theory, it is believed that in some instances, relatively high apparent release rates (for example, even greater than an apparent release rate of an agent from a physical mixture of separate homogenous particles) may be observed due to the relatively small size of the “isolated domains” contained within the particles as discussed with respect to certain embodiments of the inventions. It is believed that during dissolution, the isolated domains may be released from the particles, thereby resulting in relatively high exposures (e.g., higher surface areas) of the isolated domains, as compared to a similar homogenous particle. This effect may occur even when the entire particle does not dissolve, or has not yet dissolved. For example, portions of the particle that have not dissolved (for example, due to low solubility, and/or low rates of dissolution, e.g., CaCO₃) may become separated from the particle, thereby exposing more isolated domains to the surrounding environment. Accordingly, and surprisingly, such particles may result in apparent release rates that are higher than the apparent release rate from a similar but homogenous particle. In some cases, this may be true even if portions of the particle exhibit poor or no solubility.

As a specific illustrative non-limiting example, a test sample of particles consisting essentially of CaCO₃ and estradiol (as a specific non-limiting example of an agent) having an average particle size of about 2 micrometers may be compared to a control sample of a physical mixture of a first population of particles consisting essentially of CaCO₃ and having an average particle size of about 2 micrometers, and a second population of particles consisting essentially of estradiol and having an average particle size of about 2 micrometers, where the final ratio of the first population of particles to the second population of particles is substantially equal, by mass, to the ratio of CaCO₃ and estradiol in the particles consisting essentially of CaCO₃ and estradiol. Thus, the control sample contains the same mass ratio of CaCO₃ to estradiol as the test sample, and particles having the same sizes (or size distribution) as the test sample, but lacks the physical structure of compositions within the test sample. To determine and compare releases rates, particles in the control and test samples may be each be exposed to the same external environment, for example, to the same temperature and/or pressure, to an aqueous solution having a pH of about 5 or less, about 4 or less, about 3 or less, about 2 or less, etc. Of course, this procedure may be generalized for other compositions, including other agents such as those described herein

In some embodiments, the size of the particles may be controlled, at least in part, by the degree or vigor of mixing of the first and second fluids. For instance, rapid mixing of the first and second fluids may cause smaller particles to form, relative to slower mixing of the first and second fluids. In some cases, mixing of the first and second solutions may be controlled to produce particles having an average largest dimension of no more than about 100 micrometers, i.e., the numerical average of each of the largest dimensions of each of the particles formed is no more than about 100 micrometers. In some cases, the average largest dimension of the particles may be selected to be no more than about 50 micrometers, no more than about 30 micrometers, no more than about 10 micrometers, no more than about 5 micrometers, no more than about 3 micrometers, no more than about 1 micrometer, no more than about 500 nm, no more than about 300 nm, no more than about 100 nm, no more than about 50 nm, no more than about 30 nm, or no more than about 10 nm. In one embodiment, the particle has a largest dimension of at least about 5 nm, at least about 10 nm, at least about 30 nm, at least about 100 nm, at least about 300 nm, at least about 1000 nm, etc. The size of the particles may be determined using any suitable technique, for example, visual or electron microscopy, laser light scattering, BET, or the like.

In some cases, mixing of the first and second fluids may be performed under conditions such that the mixing time of the first and second fluids is less than about 10 s, less than about 5 s, less than about 3 s, less than about 1 s, less than about 500 ms, less than about 300 ms, less than about 100 ms, less than about 50 ms, less than about 30 ms, less than about 10 ms, less than about 5 ms, less than about 3 ms, or less than about 1 ms in some cases. The mixing of the first and second fluids may be performed, e.g., as a continuous or a batch process. For example, the first solution and the second solution may be jet-mixed or mixed in a relatively small mixer, such as a shaken vial, a vortex mixer, or a static mixer. In a jet mixer, a first stream is impinged on a second stream to cause mixing. In some cases, the mixing of the streams may be performed on a continuous basis. In some cases, relatively high fluid velocities are involved, for example, fluid velocities of at least about 10 cm/s, at least about 30 cm/s, at least about 1 m/s, at least about 3 m/s, at least about 10 m/s, at least about 30 m/s, or at least about 100 m/s at the point of contact between the first stream of fluid and the second stream. The jet mixer may be, for example, a Y or a T mixer. An example of a Y-mixer where the first and second streams intersect in a “Y” shape is discussed below. As another example, the first and second solvents may be mixed in a regime where, when the first and second solvents physically contact each other, one or both of the first and second solvents exhibits turbulent flow.

The temperature at which the fluids are contacted may be any temperature, and the temperature of the two fluids may be the same or different. For example, one or both fluids may be at ambient temperature, or in some cases, higher or lower temperatures may be used. For example, the temperature may be at least 20° C., at least about 25° C., at least about 30° C., at least about 40° C., at least about 50° C., at least about 60° C., at least about 65° C., at least about 70° C., at least about 80° C., at least about 90° C., or at least about 100° C., or the temperature may be no more than about 80° C., no more than about 70° C., no more than about 60° C., no more than about 50° C., no more than about 40° C., no more than about 35° C., no more than about 30° C., no more than about 25° C., no more than about 20° C., no more than about 15° C., or no more than about 10° C. Similarly, the pressure at which the particles are formed may be ambient pressure, or in some embodiments, higher or lower pressures may be used. For example, the formation pressure may be at least about 1.5 atm, at least about 2 atm, at least about 3 atm, at least about 4 atm, at least about 5 atm, at least about 7 atm, at least about 10 atm, etc.

In some instances, both fluids may have essentially the same temperature before mixing; in other instances, the temperature of the two fluids may differ, e.g. by more than about 2° C., more than about 5° C., more than about 10° C., more than about 20° C., more than about 40° C., more than about 70° C., more than about 100° C., more than about 150° C., etc. Accordingly, pressures of the two fluids before mixing may differ as well.

U.S. Provisional Patent Application Ser. No. 61/376,149, filed Aug. 23, 2010, entitled “Particles for Drug Delivery and Other Applications,” by Fan, et al., is incorporated herein by reference in its entirety.

The following examples are intended to illustrate certain embodiments of the present invention, but do not exemplify the full scope of the invention.

### Example 1

This example illustrates the incorporation of a hydrophobic active in CaCO₃ particles in accordance with an embodiment of the present invention. In this example, 17-beta estradiol was used as a model agent, although other agents could be used as well. 17-beta estradiol has the following structure:

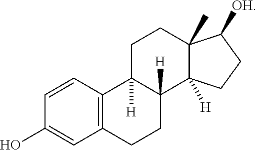

0.25 g estradiol (20 wt % to the produced CaCO₃) was dissolved in 2 ml of 0.5 M CaCl₂/EtOH. Then, 10 ml of 0.1 M Na₂CO₃/H₂O (stoichiometry of 1:1) was poured into the solution under turbulent stirring using a VWR Vortex-Genie Mixer. After 3-circle filtration (Whatman Grade No. 1 Filter Paper, Whatman 1001-070, particle retention of 11 micrometers) and washing away of excess estradiol, the particles were dissolved in 2 ml of 2% HCl/EtOH, and measured UV absorption. The washing procedure used included filtration of the particles by Buckner funnel and vacuum, then exposing the particles to 2 ml ethanol three times at room temperature, washed for about 5 seconds each time. It was found that the morphology of the particles remained substantially the same after such washing procedures.

SEM images were obtained using a Supra55VP Field Emission Scanning Electron Microscope (FESEM), which allowed surface examination down to nanometer scales in either high vacuum or in Variable Pressure (VP) mode. The SEM used a low to moderate energy (0.1 to 30 keV) electron beam to image a sample with resolutions down to 1 nm at 15 keV in high vacuum or 2 nm at 30 keV in VP mode. The Supra55VP used also included an Energy Dispersive X-ray Spectrometer (EDS) for elemental analysis (B-U) and mapping, and an Electron Back-Scattered Diffraction (EBSD) system for phase identification, crystal orientation and phase mapping using Kikuchi patterns. In these experiments, a carbon tape on metal stub was used as a substrate, with an SE2 detector and a gun voltage of 2.74 V for SEM imaging and 12 V for EDS elemental analysis. The working distance used was 10 mm for SEM imaging, and 8.5 mm for EDS elemental analysis. UV absorption was determined using a NanoDrop® ND-1000 Spectrophotometer with a 1.5 ml test solution.

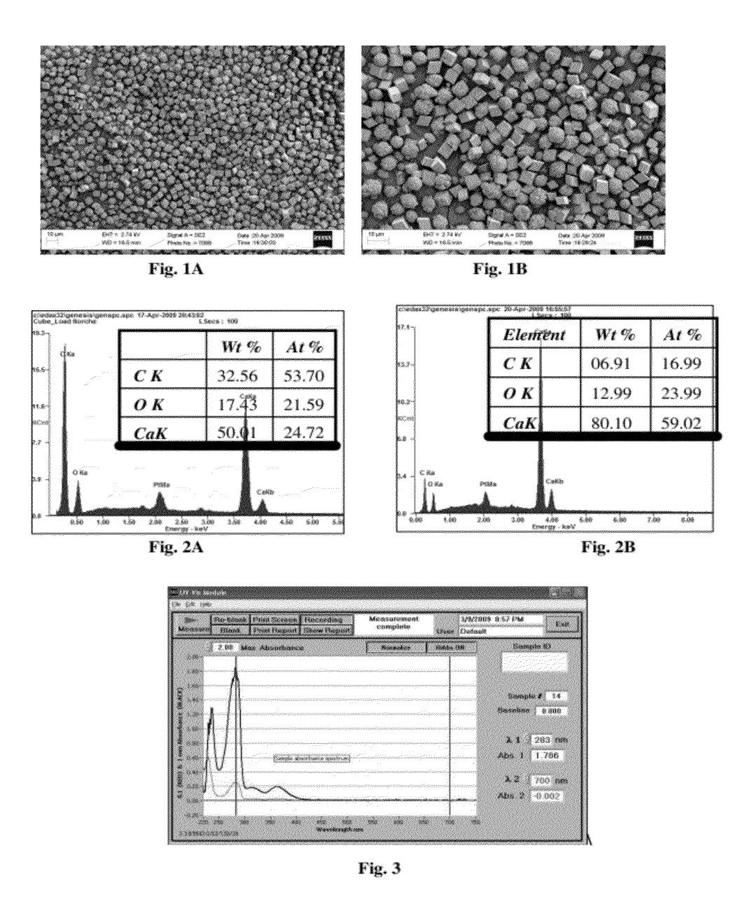

FIGS. 1A and 1B are SEM images of the particles produced using such techniques. There appeared to be two kinds of structures: clusters and rhombohedras. EDAX was used to analyze the composition of those two particles, as shown in FIG. 2. Compared to the EDAX data of pure CaCO₃ and estradiol, it appears that the rhombohedra structures were pure CaCO₃ particles, whereas the clusters appeared to be CaCO₃/estradiol hybrid particles. However, it should be noted that these results do not necessarily use optimized processing conditions, and other processing conditions may be more optimized. For example, continuous precipitation at high Reynolds numbers may be used to provide more uniform particle morphology, e.g., as discussed herein.

FIG. 3 illustrates UV the absorption of the particles dissolved as discussed above in HCl/EtOH. There is a strong peak at 283 nm in FIG. 3, which is characteristic of the absorption of 17-beta estradiol. Thus, there was a large amount of estradiol incorporated in the CaCO₃ particles.

### Example 2

In this example, Danazol was used as a model agent, although other agents may be used as well in other embodiments. Danazol has the following structure:

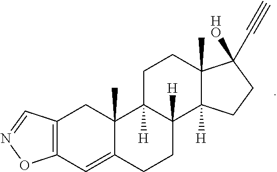

0.25 g danazol (20 wt % relative to the produced CaCO₃) was dissolved in 2 ml of 0.5 M CaCl₂/EtOH. Then, 10 ml of 0.1 M Na₂CO₃/H₂O (stoichiometry of 1:1) was poured into the solution under turbulent stirring by a vortex mixer. After 3-circle filtration and washing away of NaCl, the particles were characterized by SEM, XRD, and Raman confocal microscopy.

For the XRD measurements, a Scintag XDS2000 fixed sample position powder diffractometer was used, with a wavelength (λ) of 1.54 Angstroms and a scanning angle of between 5 and 60 degrees. The Raman confocal microscope used was a WITec Alpha-300 Confocal Raman Microscope. The microscope allowed the ability to acquire chemical information non-destructively with a resolution down to the optical diffraction limit (˜200 nm). Due to the confocal setup, it was not only possible to collect information from the sample surface, but also to examine the insides of transparent samples and also obtain 3D information. The laser used had a peak maximum of 533 nm.

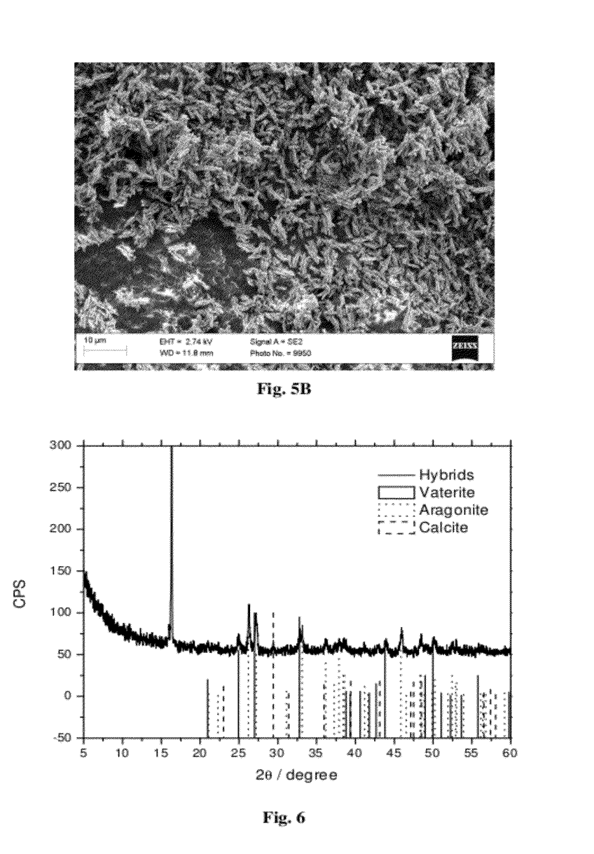

FIGS. 5A and 5B are SEM images of particles produced using such techniques. XRD was used to analyze the composition of the particles, as shown in FIG. 6. Compared to the XRD spectrums of pure calcite, aragonite, vaterite (lower lines), it was determined that these three polymorphs of CaCO₃ all were present within the particles, with greater amounts of aragonite and vaterite. There was also a strong peak at 16.3 degrees (FIG. 6), which is a characteristic of the diffraction of danazol (“hybrid,” upper trace). Using Gaussian function to fit the peak, a crystallite size of danazol was 110.8 nm calculated using the Scherrer Formula:

$${D}_{p} = \frac{0.94 λ}{{β}_{1 / 2} cos θ} .$$

In this formula, β (beta) is the line broadening at half the maximum intensity (FWHM) in radians, and θ (theta) is the Bragg angle.

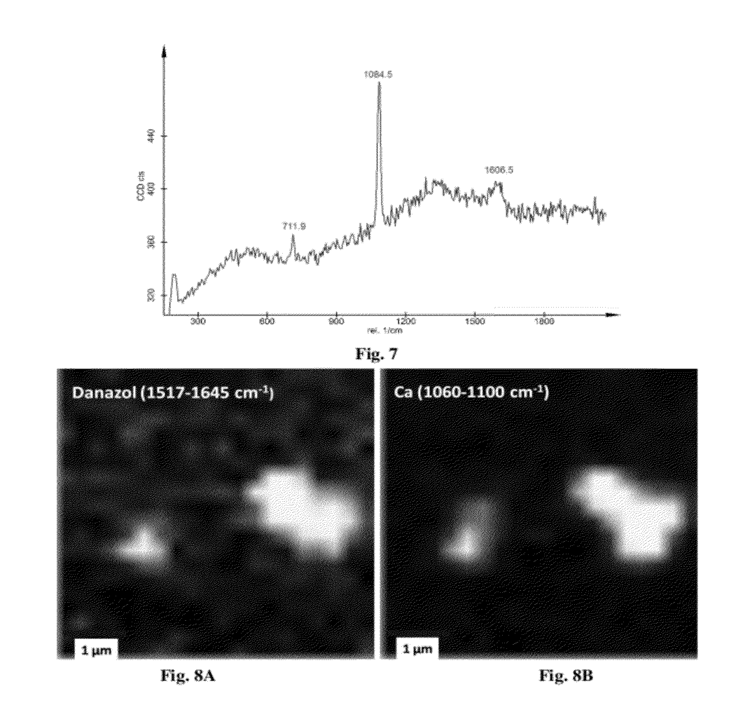

FIG. 7 illustrates a Raman spectrum of the particles produced as discussed above. There was a strong peak at 1084.5 cm⁻¹, which is generally characteristic of CaCO₃, whereas the peak at 1606.5 cm⁻¹ appears to be the carbon-carbon stretching mode of danazol. FIGS. 8A and 8B are Raman confocal layer mapping graphs of danazol and CaCO₃ respectively, showing that danazol and CaCO₃ were found to be blended together. Accordingly, based on these data, the particles appeared to include both danazol and CaCO₃.

### Example 3

This example illustrates continuous turbulent mixing to prepare particles in accordance with one embodiment of the invention. Ethanol (99+%, Sigma Aldrich) containing 0.1 M CaCl₂ (Sigma Aldrich) and 2.75 g/L of a pharmaceutically active agent (BASF) was turbulently mixed with distilled water containing 0.1 M of Na₂CO₃ (Sigma Aldrich) using a microfluidic Y-mixer (Upchurch Scientific, U-466) at a total flow rate of 3 mL per minute. The pharmaceutically active agents tested included fenofibrate, danazol, chlotrimazole, and estradiol. Within the mixer, the fluids were subjected to turbulent mixing at a Reynold's number greater than 4000. The two fluids were injected into the Y-mixer using plastic syringes (10 cc each, BD Scientific) driven by syringe pumps (Harvard Apparatus). Upon mixing, the fluid exiting the Y-mixer was collected into a stirred glass vial. Samples were collected for 2 minutes and the sample was then stirred for 5 minutes before samples of the precipitate were collected by vacuum filtration through a filter paper. Upon filtering, the filter paper containing the precipitate was dried at 65° C. for 1 hour to evaporate the remaining ethanol and water. Samples of the dried precipitate powder were collected for analysis.

### Example 4

This example illustrates particles that show relatively high apparent release rates, and techniques for determining such rates.

SEM images indicated that particles formed as previously described were composed of distinct drug and CaCO₃ domains, and that the particles are not homogenous at the molecular scale (i.e. the particle is not a molecular co-crystal). This is reasonable considering the non-polar, hydrophobic nature of the poorly water-soluble drugs and the ionic character of the calcium carbonate. Powder X-ray diffraction from the hybrids indicates that the crystal lattices of both the drugs and calcium carbonate were relatively unchanged from those of the pure precipitated compounds. In addition, the vibrational spectrum of most of the drugs appeared to be unchanged when the drugs were incorporated into the particles, as shown by FT-IR spectroscopy; this provides further evidence that the drugs do not interact strongly with the CaCO₃ when contained within the particles. In this example, the dissolution rates of the particles were determined, and compared to compared with raw, micron-sized drug crystals.

The particles in these experiments were prepared using techniques such as those discussed above (see, e.g., Examples 1 and 2). During the dissolution experiments, for convenience, dissolution media was placed in a dissolution chamber which is typically used for pressure dialysis; no pressure was applied for the measurements described here. The vessel used in these experiments contained a built-in stir bar and the dissolution media was stirred at a rate of approximately one revolution per second. Dissolution media was drawn from the bottom of the vessel, through a 0.1 micron PTFE filter. The media vessel was connected to a flow-through UV-Vis spectrophotometer cell via tubing and the liquid was driven using a peristaltic pump. A flow rate of 8 mL per second was used. The output of the flow-through cell was connected back to the dissolution chamber, so that the flow formed a closed loop; in this manner the volume of the dissolution media was conserved.

Because fenofibrate, chlotrimazole, and estradiol are poorly soluble in water, no UV-vis signal was detectable when pure water, or acidic water with added HCl was used as dissolution media. For this reason, sodium dodecyl sulfate (SDS) was added at a concentration of 10 mM. In addition, the dissolution media for most experiments had a pH of 1.5, set by the addition of HCl. All water used was distilled and de-ionized. These drugs were monitored at a wavelength of 290 nm. Templates were prepared for dissolution testing by grinding pellets in a mortar and pestle into a fine powder. The raw drug powder was not ground. All tests were performed under sink conditions, where the total amount of drug added is less than one third of the saturation concentration. All dissolution media was kept at 37° C.

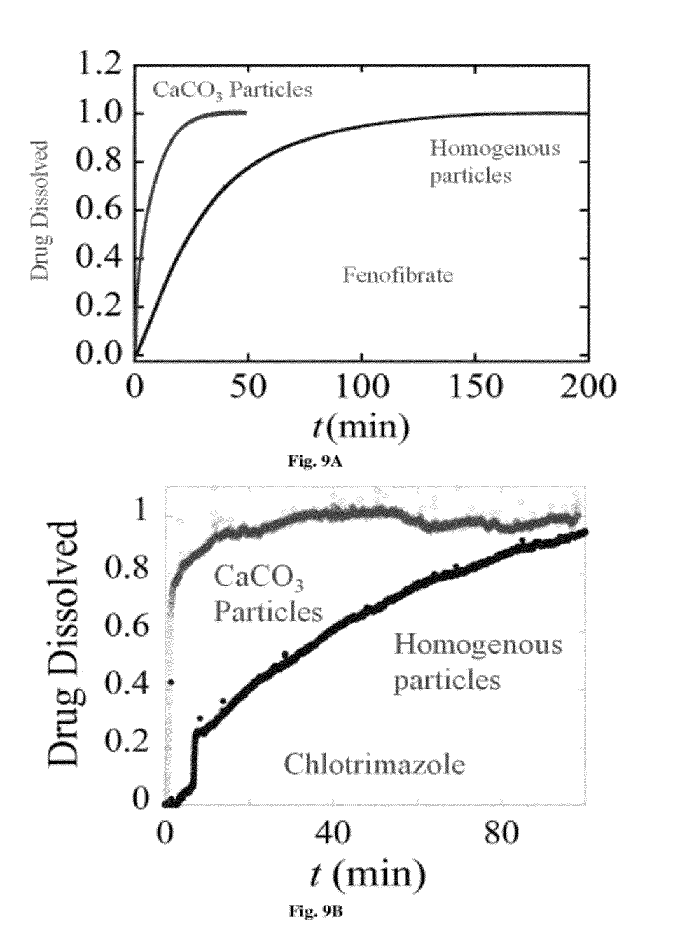

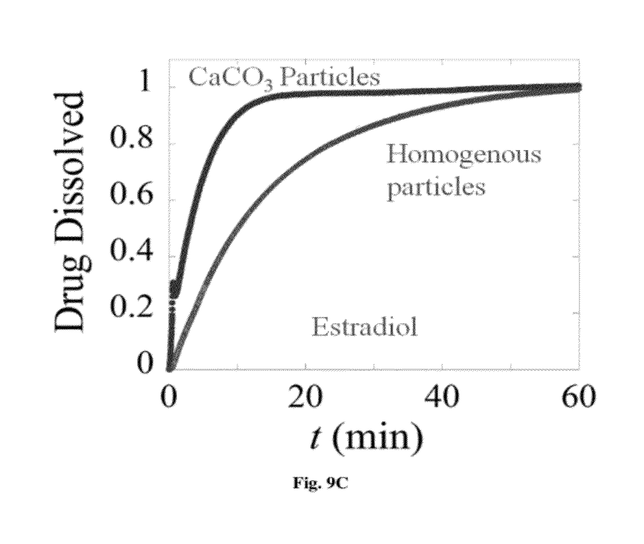

Example dissolution data using fenofibrate particles, chlotrimazole particles, and estradiol particles produced using the above protocol are shown in FIGS. 9A, 9B, and 9C, respectively. These dissolution tests indicated that drugs such as fenofibrate, danazol, chlotrimazole, and estradiol exhibited enhanced dissolution rates when incorporated into a hybrid structure, compared with raw, micron-sized drug crystals. All three experiments, the particles appeared to dissolve at apparent release rates that were much greater than the control samples.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1-4. (canceled)

5. The composition of claim 20, wherein the particle has a largest dimension of no more than about 100 micrometers.

6. The composition of claim 20, wherein the agent has a solubility in water of less than about 10 g/l at 20° C. and 1 bar.

7. The composition of claim 20, wherein at least one of the one or more carbonates is selected from the group consisting of CaCO₃, MgCO₃, H₂CO₃, NaHCO₃, Na₂CO₃, K₂CO₃, KHCO₃, or NaKCO₃.

8-14. (canceled)

15. The composition of claim 20, wherein the pharmaceutically active agent comprises estradiol.

16. The composition of claim 20, wherein the pharmaceutically active agent comprises danazol.

17-19. (canceled)

20. A composition, comprising:
a particle consisting essentially of one or more carbonates and a hydrophobic pharmaceutically active agent, wherein at least some of the agent is fluidically inaccessible from externally of the particle.

21. A composition, comprising:
a particle consisting essentially of one or more carbonates and an agent, wherein the agent is present in isolated domains within the particle, the isolated domains having a largest dimension of no more than about 1 micrometer.

22. A composition, comprising:
particles substantially each consisting essentially of one or more carbonates and an agent, wherein the particles exhibit an apparent release rate of the agent that is at least about 50% greater than an apparent release rate of the agent from a control material having the same overall composition and average diameter as the particles, wherein the control material consists essentially of chemically homogeneous particles.

23-24. (canceled)

25. A method, comprising:
providing a first fluid comprising a first solvent and a first reactant;
providing a second fluid comprising a second solvent, a second reactant, and a pharmaceutically-active agent, wherein the second fluid and the first fluid are substantially miscible; and
mixing the first fluid and the second fluid to form a mixed fluid, wherein the pharmaceutically-active agent is substantially insoluble in the mixed fluid, and wherein (a) the first reactant interacts with the second reactant to form an inorganic product that is insoluble in the mixed fluid but is soluble in aqueous-based solution at pH of less than 4, and/or (b) the first reactant interacts with the second reactant to form a non-polymerized product that is insoluble in the mixed fluid.

26. The method of claim 25, wherein the first reactant interacts with the second reactant to form an inorganic product that is insoluble in the mixed fluid but is soluble in aqueous-based solution at pH of less than 4.

27. The method of claim 25, wherein the first reactant interacts with the second reactant to form a non-polymerized product that is insoluble in the mixed fluid.

28-30. (canceled)

31. The method of claim 25, wherein the inorganic product comprises CaCO₃.

32. (canceled)

33. The method of claim 25, wherein the inorganic product and the agent co-precipitate to form a solid precipitant.

34. The method of claim 33, wherein the inorganic product and the agent co-precipitate to form a single solid precipitant.

35-40. (canceled)

41. The method of claim 25, wherein the first reactant comprises CO₃²⁻.

42-44. (canceled)

45. The method of claim 25, wherein the second reactant comprises Ca²⁺.

46-52. (canceled)

53. The method of claim 25, wherein the act of mixing comprises jet-mixing the first fluid and the second fluid.

54. The method of claim 25, wherein the act of mixing comprises mixing the first fluid and the second fluid in a mixer such that the residence time of the first fluid and the second fluid within the mixer is less than about 1 s.

55. The method of claim 25, wherein the act of mixing comprises mixing the first fluid and the second fluid such that, when the first fluid and the second fluid physically contact each other, at least one of the first fluid and the second fluid exhibits turbulent flow.

56-58. (canceled)

59. The method of claim 25, comprising mixing the first fluid and the second fluid in a Y-mixer or a T-mixer.

60-61. (canceled)
